(19) United States
(12) Patent Application Publication
(10) Pub. No.: US 2010/0300456 A1
(43) Pub. Date: Dec. 2, 2010

(54) Biohazard Wheelchair Cover

(76) Inventor: Joseph Gerard Purvis, Little Rock, AR (US)

Correspondence Address: Joseph Gerard Purvis, 319 South Pine, Little Rock, AR, 72205, US

(21) Appl. No.: 12/460,325

(22) Filed: Jul. 17, 2009

Related U.S. Application Data
(60) Provisional application No. 61/217,032, filed on May 27, 2009.

(51) Int. Cl. A61B 19/02

(52) U.S. Cl. 128/846; 128/849

(57) ABSTRACT

A wheelchair cover designed to be placed over any wheelchair to help prevent against the spread of an infectious disease from staff and patient an one patient to others.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISK APPENDIX

Not Applicable

### BACKGROUND OF THE INVENTION

The present invention is in the medical field of preventative maintenance for communicable diseases. More particularly, the present invention is in the medical field of disposable wheelchair covers.

### BRIEF SUMMARY OF THE INVENTION

The present invention is a wheelchair seating device cover as a preventative against the spread of an infectious disease from one patient to others.

### BRIEF DESCRIPTION OF THE SEVERAL VIEWS OF THE DRAWING

FIG. 1 is a perspective view of a biohazard wheelchair cover (in use) of the present invention;

FIG. 2 is a front view of the biohazard wheelchair cover of FIG. 1;

FIG. 3 is a back view of the biohazard wheelchair cover of FIG. 1.

### DESCRIPTION OF THE INVENTION

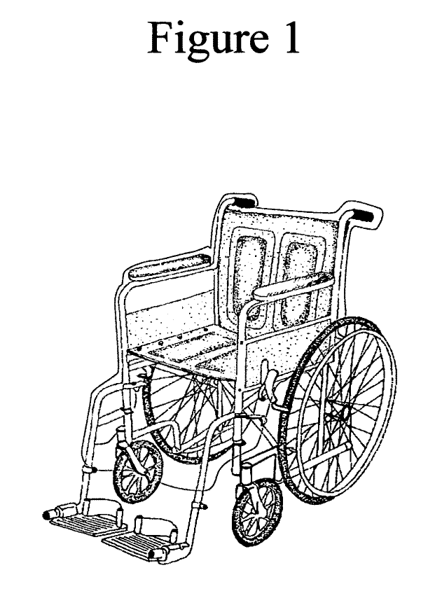

Referring now to the invention in more detail, in FIG. 1 there is shown a biohazard wheelchair cover on a wheelchair. The wheelchair cover slides over the handles and spreads across front and back surfaces of the wheelchair seat. The biohazard wheelchair cover is sufficiently wide and long enough to cover a standard wheelchair.

The construction details of the invention as shown in FIG. 1 are that the biohazard wheelchair cover is made of 0.7 mil thick, non allergenic polymers, a flexible and strong material to fit any standard size wheelchair.

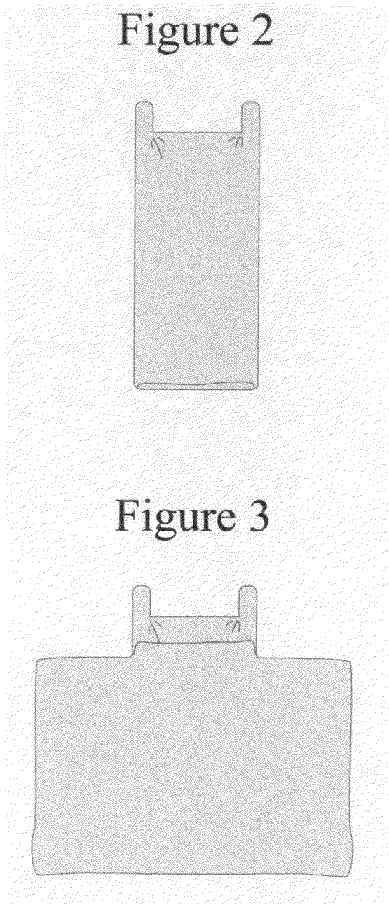

Referring now to FIG. 2 and FIG. 3, there is shown the front and back view of the invention before being placed on a wheelchair. FIG. 2 shows the folded biohazard wheelchair cover. FIG. 3 shows the back view of an unfolded biohazard wheelchair cover.

The advantages of the present invention include, without limitation that it is portable, disposable, and can be applied quickly and safely. It is easy to place the biohazard wheelchair cover over any standard size wheelchair. The invention is also easily removed for disposal so that a new uncontaminated cover can be placed over the wheelchair for the next patient.

In broad embodiment, the present invention is a biohazard cover to place over a wheelchair to protect one patient from the next against potentially infectious diseases.

The invention should therefore not be limited by the above described embodiment, method, and examples, but by all embodiments and methods within the scope and spirit of the invention as claimed.

The embodiments of the invention in which an exclusive property or privilege is claimed are defined as follows:

1. The advantages of the present invention include, without limitation that it is portable, disposable, and can be applied quickly and safely. It is easy to place the biohazard wheelchair cover over any standard size wheelchair. The invention is also easily removed for disposal so that a new uncontaminated cover can be placed over the wheelchair for the next patient.
In broad embodiment, the present invention is a biohazard cover to place over a wheelchair to protect one patient from the next and staff against potentially infectious diseases.
The invention should therefore not be limited by the above described embodiment, method, and examples, but by all embodiments and methods within the scope and spirit of the invention as claimed.
